(19) United States
(12) Patent Application Publication
(10) Pub. No.: US 2026/0108711 A1
(43) Pub. Date: Apr. 23, 2026

(54) PLASTIC STENT

(71) Applicant: UIF (University Industry Foundation), Yonsei University, Seoul (KR)

(72) Inventors: Jae Hee CHO, Seoul (KR); Sung Ill JANG, Seoul (KR); Jung Hyun JO, Seoul (KR); See Young LEE, Seoul (KR); Chan Min JUNG, Seoul (KR)

(73) Assignee: UIF (University Industry Foundation), Yonsei University, Seoul (KR)

(21) Appl. No.: 19/423,245

(22) Filed: Dec. 17, 2025

Related U.S. Application Data
(63) Continuation of application No. PCT/KR2025/011361, filed on Jul. 30, 2025.

(30) Foreign Application Priority Data
Oct. 15, 2024 (KR) 10-2024-0140463

(51) Int. Cl. A61M 27/00; A61F 2/04

(52) U.S. Cl. CPC A61M 27/008; A61F 2/04; A61F 2002/041; A61F 2002/045; A61F 2250/0098; A61M 2210/1057

(57) ABSTRACT

The present disclosure provides a plastic stent includes a front end portion formed in a tube shape and having an inlet formed on one end; a central portion extending integrally from one end of the front end portion; and a rear end portion extending integrally from the other end of the central portion and having an outlet formed at an end of a flow path communicating with the inlet, wherein the front end portion and the rear end portion provide different restoring angles or restoring forces relative to the central portion.

### CROSS-REFERENCE TO RELATED APPLICATIONS

The present application is a continuation of International Patent Application No. PCT/KR2025/011361, filed on Jul. 30, 2025, which is based upon and claims the benefit of priority to Korean Patent Application No. 10-2024-0140463 filed on Oct. 15, 2024. The disclosures of the above-listed applications are hereby incorporated by reference herein in their entirety.

### BACKGROUND

### 1. Technical Field

The present disclosure relates to a plastic tent that allows bile or phlegm to be directed into the duodenum using endoscopic retrograde cholanglo pancreatography (ERCP).

### 2. Description of Related Art

Generally, biliary stenting is an interventional procedure in the pancreatobiliary system, inserted to relieve biliary obstruction caused by biliary tract cancer or pancreatic cancer.

Biliary atresia is a narrowing or obstruction of the bile duct, a passage (duct) that carries bile produced in the liver to the duodenum, due to gallstones or tumors (malignant or benign).

When biliary atresia occurs, common diseases are associated with the location of the atresia, and using this information can aid in the differential diagnosis. Hilar biliary atresia is common in cholangiocarcinoma, gallbladder cancer, and metastatic cancer, and rarely occurs in biliary tract invasion by hepatocellular carcinoma. Obstruction of the mid-portion of the common bile duct can be caused by pancreatic cancer, cholangiocarcinoma, iatrogenic stricture, metastatic cancer, sclerosing cholangitis, or chronic pancreatitis. Common causes of distal biliary atresia include pancreatic cancer, ampullary cancer, duodenal cancer, distal bile duct cancer, and ampullary stricture.

The most characteristic symptom of biliary atresia is obstructive jaundice (severe jaundice), accompanied by upper abdominal pain, fever, and severe itching. If symptoms of biliary atresia appear, biliary drainage, dilatation, biliary stent placement, or surgical intervention are necessary to ensure proper bile flow from the biliary tract to the digestive system.

This procedure is especially necessary for patients with biliary tract and pancreatic cancer, as biliary atresia almost inevitably occurs.

Stent insertion can improve quality of life by enhancing the effectiveness of chemotherapy and radiation therapy while reducing the risk of jaundice, sepsis, and other complications. If biliary atresia occurs during chemotherapy, severe jaundice may occur. In such cases, chemotherapy must be discontinued. Therefore, biliary stent placement can improve patient symptoms and shorten treatment periods.

However, conventional stent structures are either straight or pigtailed. These stents have raised concerns that their elastic restoring force can cause damage, such as puncture or perforation of the bile duct.

### SUMMARY

The present disclosure is to provide a plastic tent that allows bile or phlegm to be directed into the duodenum using endoscopic retrograde cholanglo pancreatography (ERCP).

Technical problems of the inventive concept are not limited to the technical problems mentioned above, and other technical problems not mentioned will be clearly understood by those skilled in the art from the following description.

In an aspect of the present disclosure, a plastic stent includes a front end portion formed in a tube shape and having an inlet formed on one end; a central portion extending integrally from one end of the front end portion; and a rear end portion extending integrally from the other end of the central portion and having an outlet formed at an end of a flow path communicating with the inlet, wherein the front end portion and the rear end portion provide different restoring angles or restoring forces relative to the central portion.

The front end portion may be formed in a hook-type or half-pigtail type bent from one end of the central portion.

The front end portion may be formed with one of: a first restoration angle formed in a range of 30 to 70° from one side of the central portion, a second restoration angle formed in a range of 90 to 135°, a third restoration angle formed in a range of 135 to 180°, and a fourth restoration angle formed in a range of 180 to 270°.

The rear end portion may be formed in a pigtail shape that is twisted to overlap each other at least once.

The rear end portion may be formed in a half-pigtail shape that is bent not to overlap each other.

The front end portion may include a first marker formed of a metal material and arranged to surround the front end portion adjacent to the inlet.

The plastic stent may further include a second marker formed of a metal material and arranged at opposite ends of the central portion.

The front end portion may include a first flap formed by peeling off a portion of an outer surface of the front end portion and bent to protrude from the outer surface.

The rear end portion may include a second flap formed by peeling off a portion of an outer surface of the rear end portion and bent to protrude from the outer surface.

The front end or the rear end may have a first engaging groove formed by cutting a portion of an outer surface adjacent to the inlet or the outlet, and wherein a position thereof is adjusted by a catheter coupled to the first engaging groove.

In another aspect of the present disclosure, a plastic stent includes a front end portion formed in a tubular shape and having an inlet formed on one end; a central portion formed in a tubular shape integrally with the front end portion on one end; a rear end portion formed in a tubular shape integrally with the other end of the central portion and having an outlet formed in communication with the inlet; and a marker portion formed to surround the outer circumference of at least one or more of the front end portion, the central portion, and the rear end portion with a metal material.

The marker portion may include: a first marker positioned adjacent to the inlet of the front end portion and configured to surround an outer circumference of the front end portion; and a second marker positioned at opposite ends of the central portion and configured to surround an outer circumference of the central portion.

The front end portion and the rear end portion may provide different restoration angles or restoration forces relative to the central portion.

Specific details of other embodiments are included in the detailed description and drawings.

### BRIEF DESCRIPTION OF THE FIGURES

The above summary, as well as the detailed description of preferred embodiments of the present application described below, will be better understood when read in conjunction with the accompanying drawings. For the purpose of illustrating the present disclosure, preferred embodiments are depicted in the drawings. However, it should be understood that the present application is not limited to the precise arrangements and means illustrated.

FIGS. 1A to 1C are reference diagrams schematically illustrating a process of inserting a plastic stent according to the present disclosure.

FIG. 2 is a plan view illustrating a plastic stent according to a first embodiment of the present disclosure.

FIG. 3 is a plan view illustrating the plastic stent illustrated in FIG. 2.

FIGS. 4 and 5 are reference diagrams illustrating a delivery-integrated plastic stent.

FIG. 6 is a reference diagram illustrating the plastic stent illustrated in FIG. 3 in an unfolded state.

FIGS. 7A and 7B are reference diagrams comparing the state of the plastic stent inserted as shown in FIG. 6.

FIG. 8 is a plan view illustrating a plastic stent according to a second embodiment of the present disclosure.

FIG. 9 is a plan view illustrating a plastic stent according to a third embodiment of the present disclosure.

FIG. 10 is a plan view illustrating a plastic stent according to the fourth embodiment of the present disclosure.

FIG. 11 is a captured photograph of a procedure scene illustrating a state in which the plastic stent illustrated in FIG. 10 is being operated.

FIG. 12 is an X-ray photograph illustrating a state in which the plastic stent illustrated in FIG. 10 has been operated.

FIG. 13 illustrates the results of 4 weeks after bile duct insertion in mini-pigs of Groups 1 to 4.

### DETAILED DESCRIPTION

Hereinafter, the preferred embodiments of the present disclosure will be described in detail with reference to the accompanying drawings. The advantages and features of the present disclosure, and methods for achieving them, will become clearer with reference to the embodiments described in detail below with the accompanying drawings. However, the present disclosure is not limited to the embodiments disclosed below and may be implemented in various different forms. These embodiments are provided solely to ensure complete disclosure of the present disclosure and to fully convey the scope of the invention to those skilled in the art. The present disclosure is defined solely by the scope of the claims. The same reference numerals designate the same elements throughout the specification.

The present disclosure is susceptible to various modifications and embodiments, and specific embodiments are illustrated and described in the drawings.

However, this is not intended to limit the present disclosure to specific embodiments, but rather to encompass all modifications, equivalents, and alternatives falling within the spirit and scope of the present disclosure.

Terms including ordinal numbers, such as “first,” “second,” and the like, may be used to describe various components, but the components are not limited by these terms.

These terms are used solely to distinguish one component from another.

For example, without departing from the scope of the present disclosure, a second component may be referred to as a “first component,” and similarly, a first component may also be referred to as a “second component.”

The term “and/or” encompasses any combination of multiple related items or any of multiple related items.

When an element is referred to as being “connected” to another element, it should be understood that it may be directly connected or connected to the other element, but that other elements may also be present in between.

On the other hand, when a component is referred to as being “directly connected” to another component, it should be understood that there are no other components present in between.

The terminology used in this application is used only to describe specific embodiments and is not intended to limit the present disclosure.

The singular expression includes plural expressions unless the context clearly indicates otherwise.

In this application, terms such as “include” or “have” are intended to indicate the presence of features, numbers, steps, operations, components, parts, or combinations thereof described in the specification, but should be understood not to preemptively exclude the possibility of the presence or addition of one or more other features, numbers, steps, operations, components, parts, or combinations thereof.

Hereinafter, embodiments will be described in detail with reference to the accompanying drawings. Regardless of the drawing numbers, identical or corresponding components will be assigned the same reference numerals, and redundant descriptions thereof will be omitted.

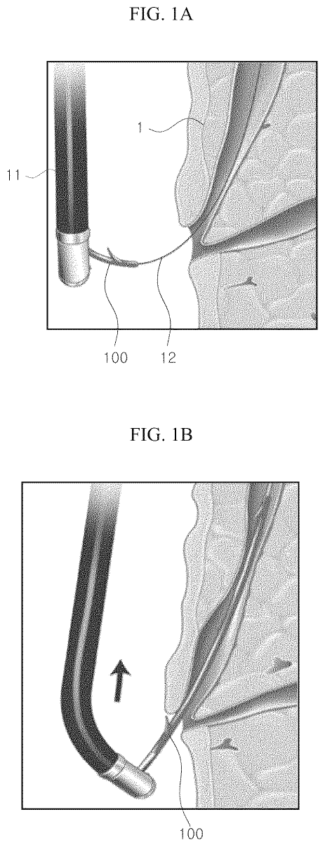

FIGS. 1A to 1C are reference diagrams schematically illustrating a process of inserting a plastic stent according to the present disclosure.

Referring to FIGS. 1A to 1C, a biliary stent may be broadly categorized into two types: endoscopically guided stent insertion and percutaneous transhepatic stent insertion. Endoscopic retrograde cholanglo pancreatography (ERCP) is commonly performed, but percutaneous transhepatic stent insertion (PTBD) may also be performed.

Endoscopic retrograde cholanglo pancreatography (ERCP) is also used as a diagnostic procedure for biliopancreatic diseases, but is primarily performed for stent insertion to treat biliary obstruction. Typically, ERCP involves opening a blocked bile duct (bile duct) through a lateral duodenoscope and inserting a drainage tube or stent into the dilated stricture.

Percutaneous transhepatic biliary stenting (PTBD) involves accessing the intrahepatic bile duct through the skin and liver parenchyma, then inserting a stent into the narrowed duct to allow bile to drain through the normal route toward the intestine. PTBD is a primary treatment for biliary atresia, using transabdominal biliary drainage from the skin when endoscopic stenting is difficult or has failed.

A stent may be broadly categorized into plastic and expandable metal materials. Each stent material has its own advantages and disadvantages, and their use may be selectively determined based on the patient's condition, surgical potential, and installation method. The present embodiment describes the plastic stent.

The major problem with stenting is the risk of occlusion due to bile sludge and tumor invasion. To prevent these problems and increase the patency of biliary stents, various materials and structures are being developed.

Here, ERCP applied in the present embodiment involves inserting an endoscope through the oral cavity into the esophagus, stomach, and duodenum under general anesthesia, and using a thin tube-shaped catheter, inserting it together with a plastic stent into the bile duct or pancreatic duct and inserting it into the stricture identified through cholangiography.

Bile produced in the liver is stored in the gallbladder, and when necessary, the gallbladder contracts to flow through the bile duct into the duodenum 3 and participate in digestion. Pancreatic juice produced in the pancreas flows through the pancreatic duct into the duodenum 3 and participates in digestion when necessary. The bile duct and pancreatic duct meet in a single tube just before entering the duodenum and connect to the duodenum 3. Here, the portion of the single tube exposed to the inner surface 1 of the duodenum is the duodenal papilla 4.

Endoscopic retrograde cholangiopancreatography is a test performed using an endoscope and x-ray. An endoscope 11 is inserted into the duodenum 3, and a contrast medium is injected into the bile duct and pancreatic duct through a hole in the duodenal papilla 4. This allows the structure of the bile duct and pancreatic duct to be examined for abnormal lesions. In addition, endoscopic retrograde cholangiopancreatography may not only confirm the structure and lesions of the bile duct and pancreatic duct, but may also collect specimens for tissue examination if necessary. It may also remove bile duct stones, drain bile, or perform a treatment with a stent 100 to treat biliary tract obstruction.

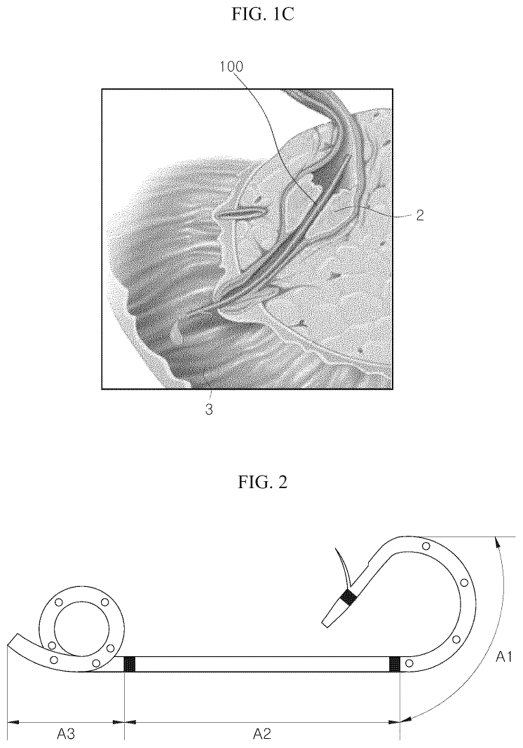

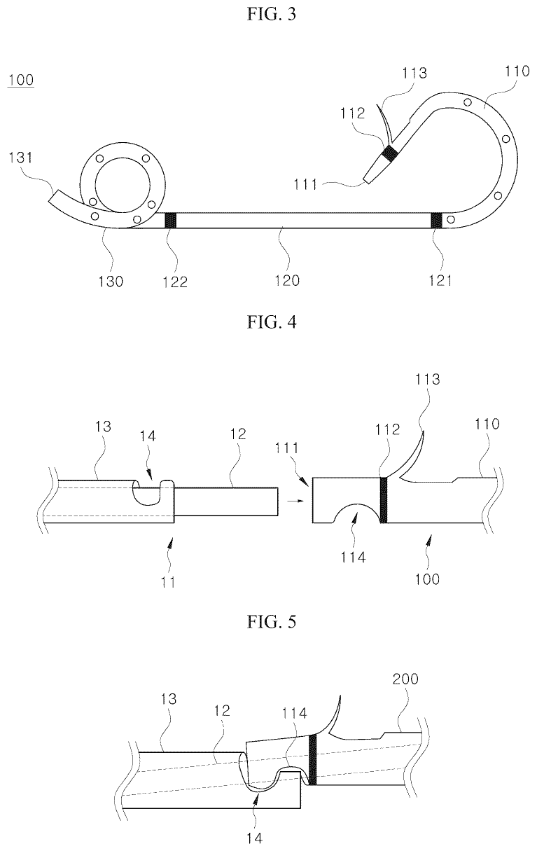

FIG. 2 is a plan view illustrating a plastic stent according to a first embodiment of the present disclosure, and FIG. 3 is a plan view illustrating the plastic stent illustrated in FIG. 2.

Referring to FIGS. 2 and 3, a plastic stent 100 according to a first embodiment of the present disclosure may include a front end portion 110, a central portion 120, and a rear end portion 130. The plastic stent 100 may be formed in a single tube structure. Here, the front end portion 110 may be positioned in a first region A1 arranged at the front of the plastic stent. In addition, the central portion 120 may be positioned in a second region A2 that is the middle of the plastic stent. In addition, the rear end portion 130 may be positioned in a third region A3 arranged at the rear of the plastic stent. Here, the front and the rear are set for the convenience of description, and the directions of the front and the rear may vary depending on the direction of progress.

First, the front end portion 110 is a portion where the plastic stent 100 first enters the duodenal papilla (4; see FIG. 1) and may be formed in a half pigtail type that is curved from one side of the central portion 120.

An inlet 111 may be formed at one end of the front end portion 110. In addition, a plurality of through holes may be formed on the outer circumferential surface of the front end portion 110. Bile or pancreatic juice may flow into the inlet 111 and the through holes. The bile or pancreatic juice flowing into the front end portion 110 may be delivered through an internal flow path that connects to the rear end portion.

In the first embodiment of the present disclosure, the front end portion 110 may be bent at an angle of approximately 180 to 270° from one end of the central portion 120. That is, when the angle reaches 180° based on a virtual extension line extending from one side of the central portion 120, the front end portion 110 may be arranged to face in the opposite direction from the central portion 120. FIGS. 2 and 3 exemplarily illustrate a case where the front end portion 110 is bent at an angle of approximately 220 to 230°.

In this case, it is preferable that the front end portion 110 be arranged so as not to overlap with the other end or the central portion 120. When the front end portion 110 is bent, it may be inserted into the bile duct in an almost straight shape, enter, and then be positioned along the shape of the bile duct by elastic restoring force.

The front end portion 110 may be provided with a first marker 112 at a set position adjacent to the inlet 111.

The first marker 112 may be provided in a ring shape made of a metal material and may be arranged to surround an outer circumferential surface of the front end portion 110. The first marker 112 may be bonded or attached to the front end portion 110, or may be integrally mounted through double injection during the molding process of the front end portion 110.

The first marker 112 is preferably made of a metal material that is free of harmful components and has excellent corrosion resistance, and may contribute to identifying the exact position of the front end portion 110 during X-ray imaging. Of course, it may also be utilized to check the degree of bending of the front end portion 110 on the plastic stent 100.

Although not shown in the drawing, the first marker 112 may be provided in the shape of multiple rings that are separated from each other. In this case, when the first marker is provided in the shape of a ring and is arranged in multiple pieces, for example, when a set number of first markers are arranged on the front end portion 110, it is expected that there will be an effect in which the position of the front end portion 110 may be quickly identified through the set number of first markers during X-ray imaging. For example, in the case that the first marker of the front end portion 110 is configured with one relatively thick ring, and the other markers provided on the central portion 120 are configured with two or three relatively thin rings, the positions of the front end portion 110 and the rear end portion 130 may be quickly identified through X-ray imaging.

The front end portion 110 may be provided with a first flap 113 adjacent to the first marker 112.

The first flap 113 may be a structure that is folded to protrude from a portion of the outer surface of the front end portion 110.

Although not illustrated in the drawing, the first flap 113 may be provided in a structure that is separately coupled without peeling off the outer surface of the front end portion 110. In this case, the first flap 113 may include a ring member (not shown) coupled to the outer surface of the front end portion 110 and a flap member (not shown) that is folded from the ring member and protrudes away from the outer surface of the front end portion.

The first flap 113 may fix the position of the front end portion 110 when the plastic stent 100 is positioned at a set installation location. For example, the first flap 113 may restrict the front end portion 110 from moving or rotating within the bile duct.

In addition, the central portion 112 may serve as a passage connecting the front end portion 110 on one side and the rear end portion 130 on the other side, and may maintain a substantially straight shape.

The length of the central portion 120 may be selectively adjusted according to gender or age.

A second marker 121 or 122 may be provided at one end and the other end of the central portion 120.

The second marker 121 or 122 may be made of a metal material to surround the outer circumferential surface of the area between the central portion 120 and the front end portion 110, and the area between the central portion 120 and the rear end portion 130.

The second marker 121 or 122 is positioned at a position distinct from the first marker 112 and may identify the center position of the plastic stent 100.

As described above, the second marker 121 or 122 may be expressed in a shape or thickness different from that of the first marker 112. In FIGS. 2 and 3, the first marker 112 and the second marker 121 or 122 are expressed in the same shape and thickness, but this is not limited thereto.

Also, although not shown in the drawing, in the case that the structure is such that the second marker 121 or 122 is wound and coupled on the outer surface of the central portion 120, a groove (not shown) may be formed along the outer surface of both ends of the central portion 120 to which the second marker 121 or 122 is coupled so as to correspond to the thickness of the second marker 121 or 122. That is, the outer surface of opposite ends of the central portion 120 may not protrude by the thickness of the second marker 121 or 122, and the outer circumferential surface of the second marker 121 or 122 and the outer circumferential surface of the central portion 120 may be formed to have the same diameter.

In addition, the rear end portion 130 may be formed with an outlet 131 located at an end of the flow path. That is, the plastic stent 1100 forms a single path from the inlet 111 to the outlet 131, so that bile or pancreatic juice may be discharged through the outlet 131 as bile or pancreatic juice flows in from the front end portion 110. Here, at least the outlet 131 of the rear end portion 130 may be exposed inside the duodenum (3; refer to FIG. 1.).

The rear end portion 130 may be formed in a roughly pigtail shape. That is, the rear end portion 130 may be formed with an area in which the end overlaps the rear end portion 130 or the central portion 120 at least once. Therefore, the rear end portion 130 may be formed with a stronger restoring force than the front end portion 110.

In addition, a through hole having the same structure as the through hole of the front end portion 110 may be formed in the rear end portion 130.

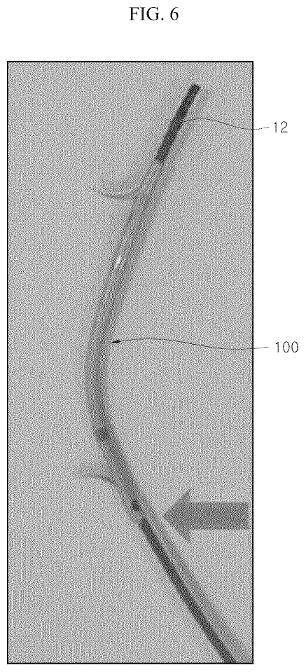

FIGS. 4 and 5 are reference diagrams illustrating a delivery-integrated plastic stent, and FIG. 6 is a reference diagram illustrating the plastic stent illustrated in FIG. 3 in an unfolded state.

Referring to FIGS. 4 to 6, a delivery-integrated plastic stent 200 may have a first engaging groove 114 formed in the front end portion 110 or the rear end portion. In the present embodiment, an example in which the first engaging groove 114 is formed adjacent to the inlet 111 of the front end portion 110 will be described.

The first engaging groove 114 may be formed by cutting approximately ⅓ to ½ of the diameter along the outer surface of the front end portion 110 so that the internal flow path is exposed on the outer surface. In this case, the first engaging groove 114 is arranged to correspond to the second engaging groove 14 of the same shape formed on a catheter 13, and the first engaging groove 114 and the second engaging groove 14 may be coupled to be interlocked with each other.

As illustrated in FIG. 5, an inner sheath 12 penetrating the inside of the catheter 13 is provided inside the catheter 13, and when the first engaging groove 114 and the second engaging groove 14 are joined and the inner sheath 12 enters the flow path of the plastic stent 200, the plastic stent 200 and the catheter 13 may maintain a bonding force. In this case, the position of the plastic stent 200 may be selectively adjusted while moving the catheter 13.

For example, the position of the plastic stent 200 may be adjusted by connecting the catheter 13 and the front end portion 110, or by connecting the catheter 13 and the rear end portion.

Once the positioning of the plastic stent 200 is completed, the inner sheath 12 may be removed from the catheter 13 to release the connection between the first engaging groove 114 and the second engaging groove 14.

Referring to FIG. 6, the plastic stent is shown with the inner sheath connected to the inside.

The inner sheath 12 may be inserted into the internal path of the plastic stent 100 and optimized for passage through a narrow stenotic area within the biliary tract (refer to FIGS. 1 and 2).

Furthermore, since the plastic stent 100 may be maintained connected to the inner sheath 12, the inner sheath 12 may be moved to a desired position in a forward and backward direction along the direction in which the plastic stent 100 is inserted.

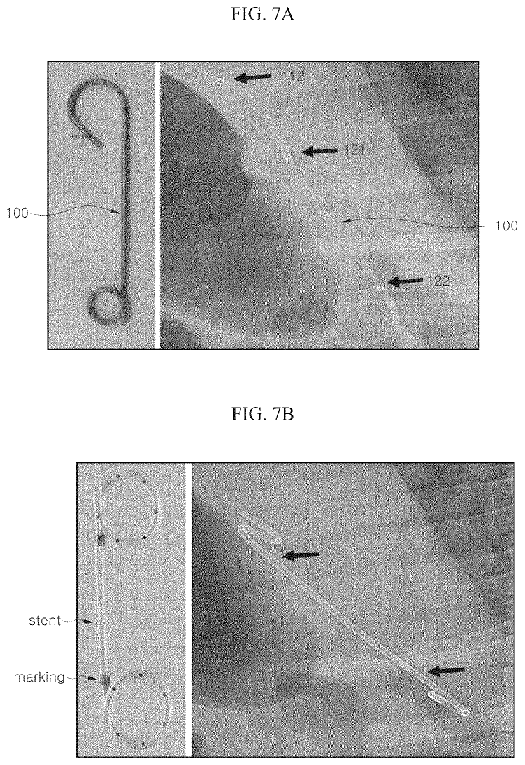

FIGS. 7A and 7B are reference diagrams comparing the state of the plastic stent inserted as shown in FIG. 6.

FIG. 7A shows an X-ray image of the plastic stent 100 according to the present disclosure inserted, and FIG. 7B shows an X-ray image of the stent inserted according to the prior art.

Referring to FIG. 7A, the plastic stent 100 of the present disclosure is provided with a plurality of metal markers 112, 121, and 122, making it easy to clearly identify the position and direction of the plastic stent 100 in the X-ray image.

In contrast, FIG. 7B has the same problem as the prior art in that it may not be identified in the X-ray image because the markings are drawn on the stent.

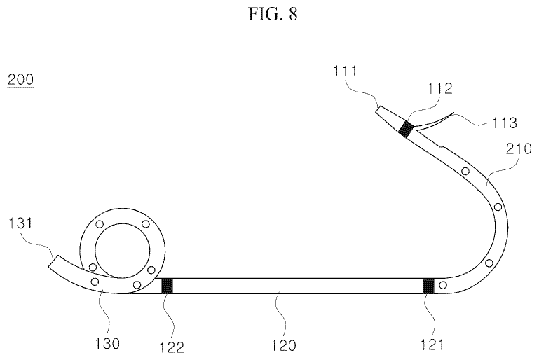

FIG. 8 is a plan view illustrating a plastic stent according to a second embodiment of the present disclosure.

Referring to FIG. 8, the plastic stent 200 according to the second embodiment of the present disclosure differs in the angle or direction of a front end portion 210. Hereinafter, the same reference numerals as those described above represent the same components.

The plastic stent 200 according to the second embodiment of the present disclosure may include a front end portion 210, the central portion 120, and the rear end portion 130.

Here, the front end portion 210 may be bent in a hook shape at an angle of approximately 135 to 180° from one side of the central portion 120. That is, it may be bent at an acute angle of at least a right angle or more based on a virtual extension line extending from one side of the central portion 120, and FIG. 8 illustrates an example of a bend of approximately 135°.

In this way, in the case that the front end portion 210 is bent by approximately 135°, the restoring force may be relatively reduced compared to the front end portion 110 of the first embodiment described above.

Therefore, there is an advantage in that the plastic stent may be selectively applied between the plastic stent 100 of the first embodiment and the plastic stent 200 of the second embodiment depending on the size of the restoring force or the shape or position of the bile duct.

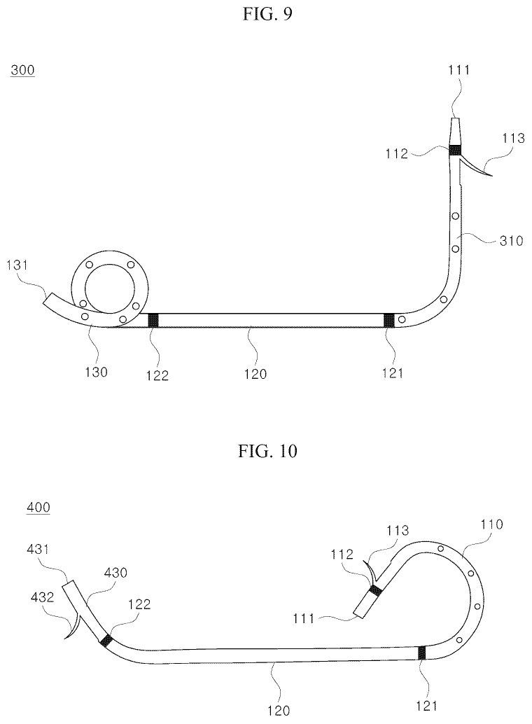

FIG. 9 is a plan view illustrating a plastic stent according to a third embodiment of the present disclosure.

Referring to FIG. 9, a plastic stent 300 according to the third embodiment of the present disclosure may include a front end portion 310, the central portion 120, and the rear end portion 130.

Here, the front end portion 310 may be bent in a longitudinal cross-section in a range of approximately 90 to 135° from one side of the central portion 120, forming an approximately “L” shape. That is, as an example, the front end portion 310 of the third embodiment is shown as being bent in a perpendicular direction based on a virtual extension line extending from one side of the central portion 120.

When the front end portion 310 is bent by approximately 90° in this way, there is an advantage in that the restoring force may be relatively reduced compared to the front end portion 210 of the second embodiment described above.

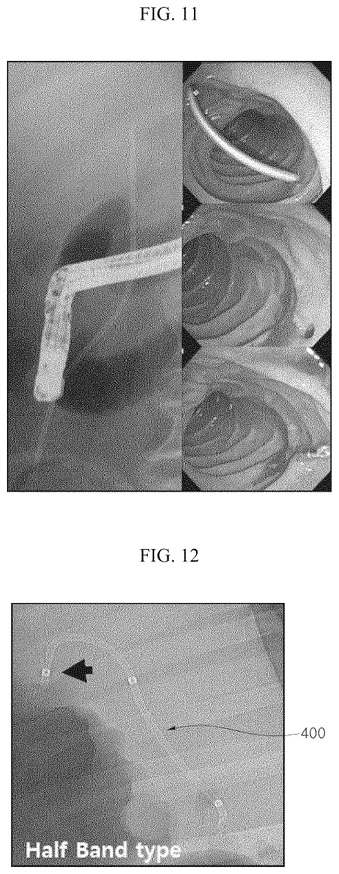

FIG. 10 is a plan view illustrating a plastic stent according to the fourth embodiment of the present disclosure, FIG. 11 is a captured photograph of a procedure scene illustrating a state in which the plastic stent illustrated in FIG. 10 is being operated, and FIG. 12 is an X-ray photograph illustrating a state in which the plastic stent illustrated in FIG. 10 has been operated.

Referring to FIGS. 10 to 12, a plastic stent 400 according to a fourth embodiment of the present disclosure may include the front end portion 110, the central portion 120, and a rear end portion 430.

Here, the front end portion 110 has the same structure as the front end portion 110 of the first embodiment described above, but there is a difference in the structure of the rear end portion 430.

Unlike the pigtail type of the first embodiment, the rear end portion 430 may be formed in a half band type.

The rear end portion 430 of the half-band type offers a structure that is approximately 50% more curved than the pigtail type, and may be bent at an angle of approximately 30 to 70° from the other side of the central portion 120, thereby significantly reducing the restoring force.

FIG. 10 illustrates a state bent approximately 70°. This half-band type rear end portion 430 structure reduces restoring force, thereby preventing biliary tract damage and stent dislodgement caused by the relatively high restoring force of the pigtail type described above.

Of course, the plastic stent 400 with the half-band type rear end portion 430 may maintain the same amount of artificial bile drainage and allows for repositioning of the surgical site, thereby increasing applicability and versatility.

In addition, the rear end portion 430 may include a second flap 432 provided on an outer surface of the rear end portion adjacent to the outlet 431.

The second flap 432 has the same shape and function as the first flap 113 provided on the front end portion 110, and provides the same effect in terms of effectiveness, so a duplicate description is omitted.

In addition, as illustrated in FIG. 11, since the rear end portion of the plastic stent is formed in a half-band type, the possibility of perforation during the process of adjusting or moving the position of the plastic stent may be significantly reduced.

For example, in the case that the rear end portion 430 has a minimal change in angle or direction with respect to the central portion 120 or is of a pigtail type, damage to the bile duct or duodenum or perforation may occur during the movement of the stent. However, a structure bent in a roughly “J” shape, such as the rear end portion of the fourth embodiment, has the advantage of significantly reducing the possibility of bile duct damage or perforation.

Although not shown in the drawing, the half-band type rear end portion 430 structure may also be applied to the front end portion 110. For example, the front end portion 110 may be bent at an angle of approximately 30 to 70° from one side of the central portion 120.

Therefore, according to the plastic stent according to an embodiment of the present disclosure, the bending direction and angle of the front and rear end portions of the stent made of plastic material may be selectively applied, and the magnitude of the restoring force may be adjusted to minimize the rate of biliary tract damage or dislocation. Furthermore, by providing a metal marker on the outer surface of the plastic stent at a set position, the position and direction of the stent may be clearly identified through X-ray or endoscopy. Furthermore, by providing a flap on the front or rear end portion, the stent may be easily fixed in place.

The following experimental examples are intended to more easily illustrate the effects of the present disclosure, and the present disclosure is not limited thereto.

### Experimental Example 1—In vitro Mechanical Performance Evaluation of Plastic Stents

In this experimental example, to evaluate the mechanical performance of the plastic stent according to an embodiment of the present disclosure, four stents with different structures are manufactured, and their mechanical properties are tested.

A total of four stents are designed identically, with an outer diameter of 7 Fr and a length of 7 cm. Their specific structures are as follows:
Group 1 (control group): No flap, no marker
Group 2 (comparative example 1: With leading flap, leading bending structure, and marker
Group 3 (comparative example 2: With trailing flap, trailing bending structure, and marker
Group 4 (one embodiment of the present disclosure): With leading and trailing flaps, leading and trailing bending structures, and marker

Mechanical property tests are conducted using ASTM-based mechanical testing equipment, and the test items included retention strength, resilience, bending strength, buckling force, and pinch friction.

The retention strength parameter measures the maximum axial resistance required by the stent to maintain its position within the bile duct or to be removed. The results show that Group 4 exhibits lower retention force compared to Groups 1 to 3, which reduce the risk of tissue damage during removal from the bile duct and ease the operator's handling burden.

The resilience parameter quantitatively measures and analyzes the tendency of the stent to return to its original shape after an external force is applied. The results show that Groups 1 to 3 exhibit high resilience, resulting in significant resistance during insertion and potentially causing tissue damage during insertion. Group 4, on the other hand, maintains adequate resilience while preventing excessive rebound, minimizing irritation to the bile duct wall.

The bending strength parameter measures the maximum strength by applying a bending load to the center of the stent. This bending strength is a key criterion for assessing stent flexibility and procedural adaptability during biliary tract insertion. Compared to Groups 1 to 3, Group 4 demonstrates the lowest bending strength, indicating its flexibility to smoothly adapt to complex anatomical pathways.

The buckling force measures the stent's resistance to buckling, a phenomenon in which the stent collapses or collapses inward when subjected to external pressure. The results show that Group 4 maintained its buckling resistance for a specified period of time, ensuring long-term patency of the drainage passage.

The pinch friction coefficient measures the coefficient of friction between the outer surface of the stent and the inner surface of the conduit or pusher system. The measurement results confirm that Group 4, due to the flap and folded structure, appropriately controls internal and external friction, enables smooth movement during insertion and removal, while preventing unnecessary migration.

Consequently, Group 4 demonstrates excellent characteristics in terms of ease of insertion, anatomical compliance, minimal irritation to the biliary tract wall, and maintenance of drainage function.

### Experimental Example 2—In vivo Evaluation of a Biliary Stricture Animal Model

To evaluate structural stability and tissue response in biliary stricture conditions, preclinical animal testing is conducted using minipigs (approximately 30 kg). A total of 16 pigs are subjected to radiofrequency ablation (RFA) to induce strictures in the common bile duct. After insertion in Group 4, the stent is evaluated for stent displacement, histological reactions (inflammation, fibrosis, epithelial damage, etc.), sludge and biofilm formation, and luminal patency over a 4-week observation period.

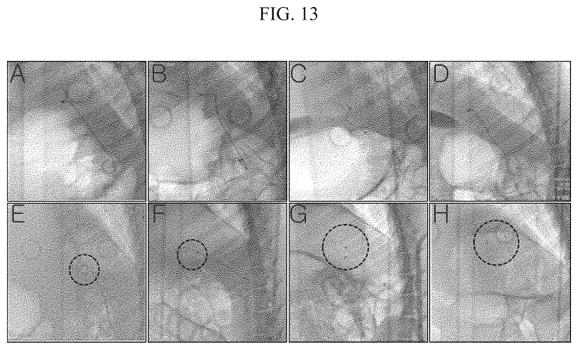

FIG. 13 illustrates X-ray pictures showing whether the position is maintained 4 weeks after bile duct insertion in minipigs of Groups 1 to 4. For reference, FIG. 13A is a picture of group 1 inserted, FIG. 13E is a picture of group 1 and 4 weeks after insertion, FIG. 13B is a picture of group 2 inserted, FIG. 13F is a picture of group 2 and 4 weeks after insertion, FIG. 13C is a picture of group 3 inserted, FIG. 13G is a picture of group 3 and 4 weeks after insertion, FIG. 13D is a picture of group 4 inserted, and FIG. 13H is a picture of group 4 and 4 weeks after insertion.

Referring to FIG. 13, Group 4 shows no cases of biliary tract dislocation in the minipigs. Compared to Groups 1 to 3, the tissue reaction score is significantly lowered. Lymphocyte infiltration and epithelial damage are minimal throughout the entire insertion period. Scanning electron microscopy (SEM) reveals that the lumen diameter is maintained at its widest, and sludge accumulation is minimal.

### Experimental Example 3—Clinical Application

When Group 4 is applied to patients with biliary stenosis following liver transplantation, the instability of the insertion site and the bile duct dilatation that occurred with conventional stents are resolved. Three-month imaging confirms the clinical efficacy of the stent, which maintained its shape and position stably.

Although specific embodiments have been illustrated and described above to illustrate the technical idea of the present disclosure, the present disclosure is not limited to the same configuration and operation as the specific embodiments described above, and various modifications may be implemented without departing from the scope of the present disclosure. Therefore, such modifications should also be considered to fall within the scope of the present disclosure, and the scope of the present disclosure should be determined by the claims set forth below.

According to an embodiment of the present disclosure, a plastic stent can, firstly, selectively apply the bending direction or angle of the front end portion and rear end portion of a stent made of plastic material.

Secondly, by controlling the magnitude of the restoring force, the rate of biliary tract damage or dislocation can be minimized.

Thirdly, by providing a metal marker on the outer surface of the plastic stent at a set position, the position or direction of the stent can be clearly identified through X-ray or endoscopy.

Fourthly, by providing a flap on the front end portion or rear end portion, the stent can be easily fixed in position.

The effects of the present disclosure are not limited to the effects mentioned above, and other effects not mentioned can be clearly understood by those skilled in the art from the description.

What is claimed is:

1. A plastic stent comprising:
a front end portion formed in a tube shape and having an inlet formed on one end;
a central portion extending integrally from one end of the front end portion; and
a rear end portion extending integrally from the other end of the central portion and having an outlet formed at an end of a flow path communicating with the inlet,
wherein the front end portion and the rear end portion provide different restoring angles or restoring forces relative to the central portion.

2. The plastic stent of claim 1,
wherein the front end portion is formed in a hook-type or half-pigtail type bent from one end of the central portion.

3. The plastic stent of claim 2,
wherein the front end portion is formed with one of:
a first restoration angle formed in a range of 30 to 70° from one side of the central portion,
a second restoration angle formed in a range of 90 to 135°,
a third restoration angle formed in a range of 135 to 180°, and
a fourth restoration angle formed in a range of 180 to 270°.

4. The plastic stent of claim 1,
wherein the rear end portion is formed in a pigtail shape that is twisted to overlap each other at least once.

5. The plastic stent of claim 1,
wherein the rear end portion is formed in a half-pigtail shape that is bent not to overlap each other.

6. The plastic stent of claim 1,
wherein the front end portion includes a first marker formed of a metal material and arranged to surround the front end portion adjacent to the inlet.

7. The plastic stent of claim 1, further comprising a second marker formed of a metal material and arranged at opposite ends of the central portion.

8. The plastic stent of claim 1,
wherein the front end portion includes a first flap formed by peeling off a portion of an outer surface of the front end portion and bent to protrude from the outer surface.

9. The plastic stent of claim 1,
wherein the rear end portion includes a second flap formed by peeling off a portion of an outer surface of the rear end portion and bent to protrude from the outer surface.

10. A plastic stent according to claim 1,
wherein the front end or the rear end has a first engaging groove formed by cutting a portion of an outer surface adjacent to the inlet or the outlet, and wherein a position thereof is adjusted by a catheter coupled to the first engaging groove.

11. A plastic stent comprising:
a front end portion formed in a tubular shape and having an inlet formed on one end;
a central portion formed in a tubular shape integrally with the front end portion on one end;
a rear end portion formed in a tubular shape integrally with the other end of the central portion and having an outlet formed in communication with the inlet; and
a marker portion formed to surround the outer circumference of at least one or more of the front end portion, the central portion, and the rear end portion with a metal material.

12. The plastic stent of claim 11,
wherein the marker portion includes:
a first marker positioned adjacent to the inlet of the front end portion and configured to surround an outer circumference of the front end portion; and
a second marker positioned at opposite ends of the central portion and configured to surround an outer circumference of the central portion.

13. The plastic stent of claim 11,
wherein the front end portion and the rear end portion provide different restoration angles or restoration forces relative to the central portion.

14. The plastic stent of claim 11,
wherein the front end portion is formed with one of:
a first restoration angle formed in a range of 30 to 70° from one side of the central portion,
a second restoration angle formed in a range of 90 to 135°,
a third restoration angle formed in a range of 135 to 180°, and
a fourth restoration angle formed in a range of 180 to 270°.

15. The plastic stent of claim 11,
wherein the front end portion includes a first flap formed by peeling off a portion of an outer surface of the front end portion and bent to protrude from the outer surface.

16. The plastic stent of claim 11,
wherein the rear end portion includes a second flap formed by peeling off a portion of an outer surface of the rear end portion and bent to protrude from the outer surface.

17. A plastic stent according to claim 11,
wherein the rear end portion includes an engaging groove formed by cutting a portion of an outer surface adjacent to the inlet, and a position there of is adjusted by a catheter coupled to the engaging groove.
